(19) United States
(12) Patent Application Publication
(10) Pub. No.: US 2012/0277321 A1
(43) Pub. Date: Nov. 1, 2012

(54) METHOD FOR PREPARING A COMPOSITION COMPRISING A COMPOUND BASED ON VANILLIN AND ETHYL VANILLIN, RESULTING COMPOSITION AND USES THEREOF

(76) Inventors: Jean-Claude Le-Thiesse, Saint-Etienne (FR); Kilani Lamiri, L'arbresle (FR)

(21) Appl. No.: 13/500,747

(22) PCT Filed: Oct. 1, 2010
(86) PCT No.: PCT/EP2010/064641

§ 371 Date: Jun. 26, 2012

(30) Foreign Application Priority Data
Oct. 8, 2009 (FR) 09 04810

(51) Int. Cl. A23L 1/22; A61K 8/34; A61K 47/36; A61K 8/65; A61Q 13/00; A61K 8/92; A61K 47/38; A61K 47/42; A61K 47/10; A61K 8/73

(52) U.S. Cl. 514/770; 514/772; 512/1; 514/777; 514/778; 514/781; 514/774; 426/534

(57) ABSTRACT

A method for preparing a composition including a compound based on vanillin and ethyl vanillin is described. A resulting composition and uses thereof in many fields of application, in particular in human and animal food is also described. A method for preparing a composition including a compound based on vanillin and ethylvanillin in a vanillin/ethyl vanillin molar ratio of 2 is also described wherein the method includes: a step of melting a mixture of vanillin and ethyl vanillin, which are used in a molar ratio other than 2, with an excess of vanillin representing from 2% to 20% of the weight of the mixture; a step of solidifying same, by cooling to a temperature of less than or equal to 50° C.±1° C.; and a step of recovering the resulting composition including the new compound.

The present invention relates to a process for preparing a composition comprising essentially a compound based on vanillin and ethyl vanillin.

The invention also relates to the resulting composition and to the uses thereof in many fields of application, in particular in human food and animal feed.

Vanillin or 4-hydroxy-3-methoxybenzaldehyde is a product widely used in many fields of application as a flavoring and/or fragrance.

Thus, vanillin is consumed abundantly in the food and animal-feed industry, but it also has applications in other fields, such as, for example, pharmacy or perfumery. Consequently, it is a product with a high level of consumption.

Vanillin is very often combined with ethyl vanillin or 3-ethoxy-4-hyrdoxybenzaldehyde, since it is known that the presence of a small amount of ethyl vanillin makes it possible to intensify the fragrancing and/or organoleptic properties of vanillin.

Thus, a potential user would like to be provided with a ready-made mixture of vanillin and ethyl vanillin.

The problem that arises is that preparing said mixture by means of a conventional technique of dry mixing of vanillin and ethyl vanillin powders results in the production of a mixture which is very liable to cake. As a result, it is impossible to use such a mixture owing to its presentation, which is not in pulverulent form, and to very great difficulty in solubilizing the mass obtained.

Moreover, prolonged storage leads to a worsening of the caking phenomenon, resulting in the powder setting.

Thus, it is desirable to have available a new presentation in solid form, based on vanillin and ethyl vanillin, which has improved flowability properties and an absence of caking on storage.

The applicant has found, according to French patent application No. 08 05913, that a new compound obtained by co-crystallization of vanillin and ethyl vanillin used in a vanillin/ethyl vanillin molar ratio of 2, exhibits unique properties, in particular with regard to its flowability properties and its lack of caking.

Said compound is in the form of a white powder which has a melting point, measured by differential scanning calorimetry, of 60° C.±2° C., different than that of vanillin and ethyl vanillin, of 81° C.±1° C. and 76° C.±1° C., respectively.

It has its own specific X-ray diffraction spectrum, which is different than that of vanillin and ethyl vanillin.

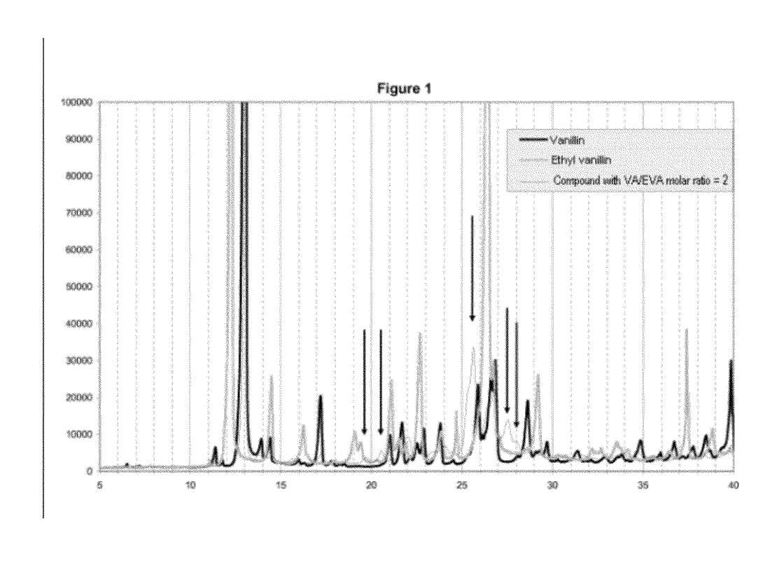

FIG. 1 shows three curves corresponding to the various X-ray diffraction spectra of the new compound of vanillin and ethyl vanillin, of vanillin and of ethyl vanillin.

On the spectrum of the new compound of vanillin and ethyl vanillin, the presence of lines at angles 2θ (°)=20.7-25.6-27.5-28.0 is in particular noted; said lines being absent from the X-ray diffraction spectra of vanillin and of ethyl vanillin.

Another characteristic of said compound is that its X-ray diffraction spectrum does not undergo any significant modification during prolonged storage.

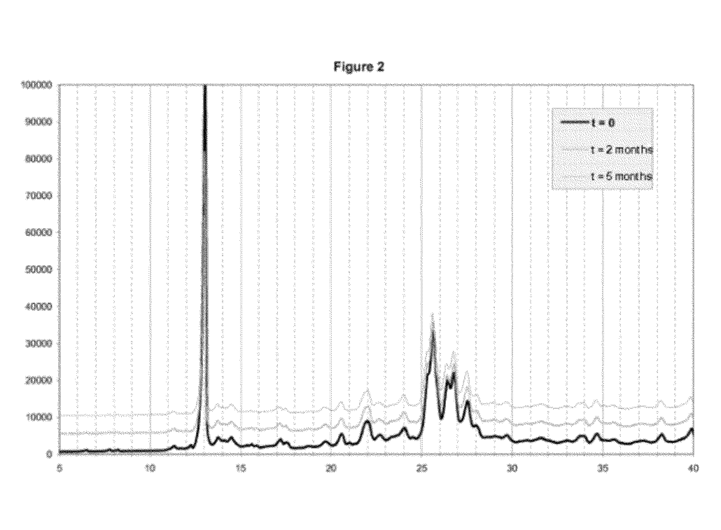

The change in its spectrum was monitored as a function of the storage time at ambient temperature. Over a prolonged storage period (five months), absolutely no modification of the spectrum of the new compound is observed, as demonstrated in FIG. 2.

FIG. 2 shows the change in the X-ray diffraction spectrum of the new compound, as a function of the storage time. It shows three curves corresponding to the various X-ray diffraction spectra of the compound of the invention obtained at time t=0, and then after storage for two months and five months.

The three curves obtained are normally superimposed. In order to be able to distinguish them better, two of these three curves of FIG. 2 have a base line that is intentionally shifted relative to the reference base line, which is the X-ray diffraction spectrum at time t=0. The curve corresponding to the X-ray diffraction spectrum obtained after storage for two months is shifted by 5000 counts/s and that obtained after storage for five months is shifted by 10 000 counts/s.

FIG. 2 demonstrates that there is no change in the compound of the invention after prolonged storage.

An absence of modification of the specific lines of the new compound of vanillin and ethyl vanillin with a vanillin/ethyl vanillin molar ratio of 2 is noted.

Another characteristic of said compound is that it is a compound that is not or very sparingly hygroscopic like vanillin and ethyl vanillin.

The hygroscopicity of said compound is determined by measuring its weight change after having been kept at 40° C. for 1 hour under air at 80% relative humidity.

Said compound adsorbs less than 0.5% by weight of water, and its content is preferably between 0.1 and 0.3% by weight of water. Said compound remains perfectly solid.

Moreover, this compound has good organoleptic properties and it possesses a high aromatic power which is far greater than that of vanillin.

Thus, the compound as defined and which is denoted in the remainder of the text “new compound” has specific properties which are reflected by a reduced ability to cake compared with a composition of vanillin and ethyl vanillin obtained by simple dry mixing.

The particular properties of the compound based on vanillin and ethyl vanillin as previously described are linked to two parameters, namely the molar ratio between the vanillin and the ethyl vanillin and the fact that there is co-crystallization between the vanillin and ethyl vanillin in a specific crystalline form characterized by its melting point and its X-ray diffraction spectrum.

One of the routes for obtaining said compound lies in a process which consists in melting the mixture of vanillin and ethyl vanillin used in a molar ratio of 2, then cooling the molten mixture by reducing the temperature to 50°±1° C., and then maintaining this temperature until the mixture has completely solidified.

The cooling is advantageously carried out in the absence of any stirring.

To this effect, the vanillin and the ethyl vanillin used in a molar ratio of 2 are loaded separately or as a mixture, and the mixture is brought to a temperature which is selected between 60° C. and 90° C. and which is preferably between 70° C. and 80° C.

It is desirable to carry out the preparation of this molten mixture under an atmosphere of inert gas, which is preferentially nitrogen.

The mixture is kept at the selected temperature until the molten mixture is obtained.

The molten product is transferred into any container, for example a stainless steel tray that will allow easy recovery of the product after solidification. This container is preheated to between 70 and 80° C. before it receives the molten mixture.

In a subsequent step, the molten mixture is cooled to a temperature of 50° C.±1, by controlling the cooling temperature by any known means.

As mentioned previously, the cooling is preferably carried out in the absence of any stirring.

The solidified mixture obtained can then be formed according to various techniques, in particular milling.

This process therefore makes it possible to obtain the new compound of vanillin and ethyl vanillin, but it has the disadvantage of not being readily transposable to the industrial scale since the crystallization of the compound is quite slow. This is because said compound exhibits a supercooling phenomenon, i.e. when the product is molten and it is cooled below its melting point, it crystallizes with difficulty and remains in the liquid state for a long time. The time required for the crystallization is more or less random and it is important to correctly control the crystallization.

Thus, cooling to a temperature of less than 50° C.±1, for example 20° C., makes it possible to accelerate the process of solidification of the molten mixture, but the crystallization is heterogeneous with the coexistence of various crystalline phases, some of which are unstable at ambient temperature or very hygroscopic. This results in considerable caking on storage of a vanillin−ethyl vanillin mixture crystallized under such conditions.

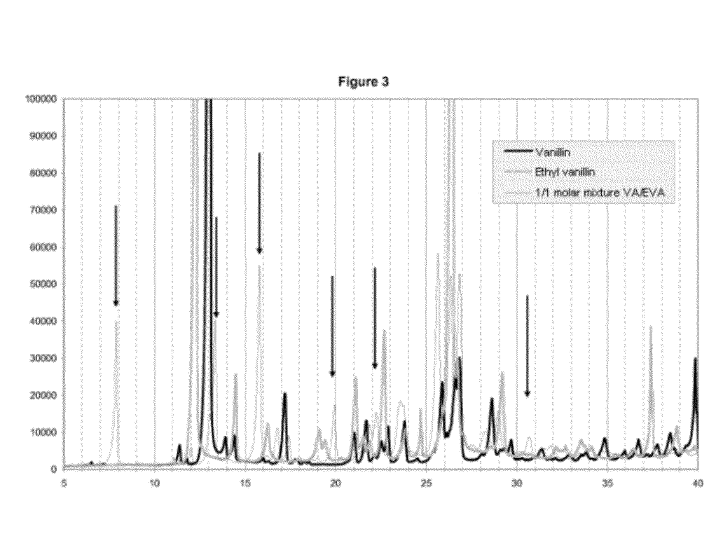

By way of comparative example, in order to illustrate the importance of the vanillin−ethyl vanillin molar ratio and the conditions for crystallization of the molten mixture, FIG. 3 represents the X-ray diffraction spectrum of an equimolar vanillin−ethyl vanillin mixture, melted at 70° C., then crystallized by rapid cooling to 20° C.

This spectrum is different than that of vanillin, than that of ethyl vanillin and than that of the new compound of vanillin and ethyl vanillin with a vanillin/ethyl vanillin molar ratio of 2, with specific lines in particular at angles 2θ (°)=7.9-13.4-15.8-19.9-22.2-30.7.

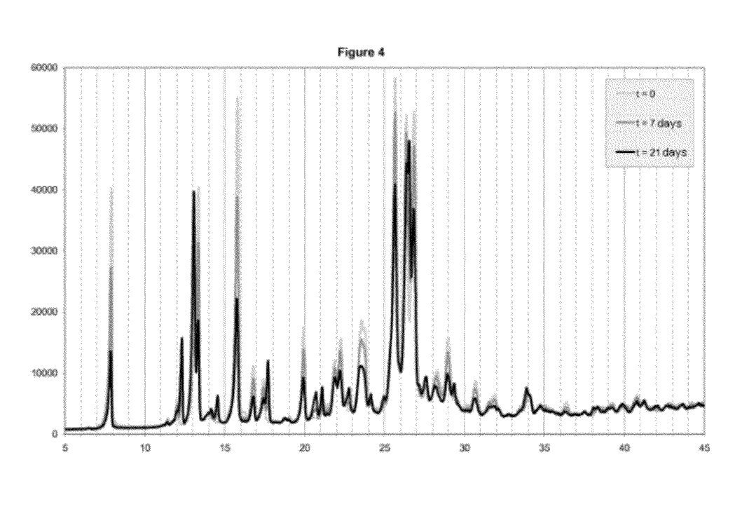

FIG. 4 shows the change in this spectrum over a storage period of three weeks at 22° C., proving that the phases thus crystallized are unstable and change rapidly while causing caking of the product.

This product has a melting point of 48° C.±1 and is found to be very hygroscopic: over the course of 1 hour at 40° C. and under air at 80% relative humidity, it adsorbs more than 4% of water by weight and becomes deliquescent.

Its properties are therefore very different than those of the new compound as previously described and do not make it possible to solve the caking problems posed by vanillin−ethyl vanillin mixtures.

The objective of the present invention is to provide a process transposable to the industrial scale, which makes it possible to obtain essentially the new compound of vanillin and ethyl vanillin with a vanillin/ethyl vanillin molar ratio of 2.

Another objective of the invention is that it results in a composition comprising same, which has the improved properties as mentioned above.

There has now been found, and it is this which constitutes the subject of the present invention, a process for preparing a composition comprising essentially a compound based on vanillin and ethyl vanillin in a vanillin/ethyl vanillin molar ratio of 2, characterized in that it comprises:
a step of melting a mixture of vanillin and ethyl vanillin, which are used in a molar ratio other than 2, with an excess of vanillin representing from 2 to 20% of the weight of the mixture: the melting temperature being selected such that the new compound obtained is completely molten but that the excess vanillin remains in the solid state finely dispersed in the molten mixture in order to act as crystallization seeds,
a step of solidifying by cooling to a temperature of less than or equal to 50° C.±1° C.,
a step of recovering the resulting composition comprising the new compound,
optionally, a step of heat treatment to a temperature of 51° C.±1° C.

In the present text, the expression “composition comprising essentially a compound based on vanillin and ethyl vanillin” is intended to mean a composition comprising at least 80% by weight of a mixture of the new vanillin/ethyl vanillin compound with a vanillin/ethyl vanillin molar ratio of 2 and of vanillin: the vanillin representing less than 25% by weight of said mixture.

The expression “new vanillin/ethyl vanillin compound” is intended to mean the compound in anhydrous form and hydrates thereof.

In accordance with the invention, it has been found that the new compound of vanillin and ethyl vanillin is readily obtained provided that its crystallization is carried out in the presence of an excess of vanillin. Under these conditions, the new compound solidifies rapidly.

The applicant has found that the presence of an excess of vanillin can act as crystallization seeds and thus facilitate the crystallization of the new compound.

In order to ensure an excess of vanillin relative to the molar ratio of 2, the vanillin and the ethyl vanillin are used in the following proportions:
from 67 to 72% by weight of vanillin,
from 28 to 33% by weight of ethyl vanillin.

In accordance with a preferred mode of the invention in which a small excess of vanillin is preferred, the proportions are advantageously the following:
from 67 to 70% by weight of vanillin,
from 30 to 33% by weight of ethyl vanillin.

In accordance with the process of the invention, an operation is carried out which consists in melting the new compound while keeping the excess vanillin in the solid state.

To this effect, the vanillin and the ethyl vanillin are loaded separately or as a mixture and the mixture is brought to a temperature which is selected such that the new compound of vanillin and ethyl vanillin is in the molten state, whereas the excess vanillin is not molten.

As previously mentioned, the melting temperature for the new compound is selected above the temperature of the new compound, that is to say 60° C.±2° C., but below the melting temperature of the excess vanillin.

Preferably, the melting temperature is chosen between 62° C. and 70° C., preferably between 62° C. and 65° C. This temperature range is given for dry powders (less than 0.2% water).

This operation is generally carried out with stirring in any device, in particular in a tank equipped with a conventional heating device such as, for example, a system of heating via electrical resistances or else via circulation of a heat-transfer fluid in a double jacket or else in a heated chamber such as a furnace or stove.

It is desirable to perform this melting under an atmosphere of inert gas, which is preferentially nitrogen.

According to one variant of the process, the excess vanillin can be introduced at the end of the melting step.

In this case, the vanillin and the ethyl vanillin are loaded separately or as a mixture in a molar ratio of 2 (65% by weight of vanillin and 35% by weight of ethyl vanillin) and then this mixture is kept at the selected temperature until the mixture is completely molten.

The excess vanillin, representing from 2 to 20% of the weight of the mixture, is then added to the molten mixture and finely dispersed by stirring.

In a subsequent step, the molten mixture is cooled to a temperature of 50° C.±1, by controlling the cooling temperature by any known means.

According to one preferred variant of the process of the invention, the cooling is preferably carried out in the absence of any stirring.

At this stage, an entirely original composition is obtained.

It is in the form of a dispersion of vanillin in the new compound of vanillin and ethyl vanillin with a vanillin/ethyl vanillin molar ratio of 2.

The compound obtained according to the process of the invention comprises at least 80% by weight, preferably at least 90% by weight of a mixture of the new vanillin/ethyl vanillin compound and of vanillin.

The composition obtained comprises less than 20% by weight, preferably less than 10% by weight of other crystalline phases of the vanillin/ethyl vanillin phase diagram and optionally of ethyl vanillin: this mixture subsequently being denoted “other crystalline phases”.

More specifically, the compositions obtained may comprise:
from 80 to 99% by weight of a mixture of the new vanillin/ethyl vanillin compound and of vanillin,
from 1 to 20% by weight of other crystalline phases.

The preferred compositions of the invention comprise:
from 90 to 99% by weight of a mixture of the new vanillin/ethyl vanillin compound and of vanillin,
from 1 to 10% by weight of other crystalline phases.

In the mixture obtained which comprises the new vanillin/ethyl vanillin compound and vanillin, the vanillin represents less than 20% by weight, preferably less than 14% by weight of said mixture.

More specifically, the mixtures obtained may comprise:
from 80 to 94% by weight of the new vanillin/ethyl vanillin compound,
from 6 to 20% by weight of vanillin.

The preferred mixtures have the following composition:
from 86 to 94% by weight of the new vanillin/ethyl vanillin compound,
from 6 to 14% by weight of vanillin.

The process of the invention therefore results in a solidified composition which can be formed, and various techniques can be envisioned.

One of them consists in milling the resulting mixture such that the particle size is compatible with the application envisioned.

It most commonly extends between 100 μm and 2 mm.

Generally, the particle size, expressed by the median diameter (d₅₀), ranges from 100 μm to 800 μm, preferably between 200 μm and 300 μm. The median diameter is defined as being such that 50% by weight of the particles have a diameter greater than or less than the median diameter.

The milling operation can be carried out in a conventional apparatus, such as a blade mill, a toothed roll crusher or a granulator.

Another forming can be carried out using the technique of flake formation on a drum or belt.

A molten mixture of vanillin and ethyl vanillin is prepared in the proportions and under the operating conditions previously indicated. The molten mixture is then brought into contact with a metal drum or belt cooled to a temperature of 50° C., and then the film obtained on the drum is scraped with a blade, to recover the solid mixture of vanillin and ethyl vanillin in the form of flakes.

It is also possible to carry out the forming according to a prilling technique in a device in which the molten mixture of vanillin and ethyl vanillin is dispersed in the form of droplets in a stream of air, preferably oxygen-depleted air, for example by dropping it from the top of a tower in a column of cold air, which results in a solid product being obtained in the form of drops a few hundred microns in diameter.

The forming may also be carried out by spray-cooling. The molten mixture of vanillin and ethyl vanillin is sprayed in the form of droplets, in a stream of cold air, preferably oxygen-depleted air, by virtue of which the solid product is obtained in the form of beads a few tens of microns in diameter.

Depending on the forming technique used, it may be more or less easy to apply a crystallization temperature rigorously equal to 50° C.±1; this is, for example, the case for prilling or spray-cooling.

A variant of the process then consists in crystallizing the molten mixture at any temperature below 50° C., preferably between 20° C. and 50° C. (limit excluded), in recovering the resulting solid and then in subjecting it to a heat treatment known as an “annealing operation”.

This annealing is carried out by gradually bringing the solid obtained to a temperature of 51° C.±1 and keeping it at this temperature for several minutes. Preferably, this annealing is carried out with stirring, for example in a mixer or in a fluidized bed.

In the same way, the melting temperature selected between 62° C. and 65° C. is understood to be for perfectly dry vanillin and ethyl vanillin powders.

Depending on the storage conditions for these products, they may be slightly moist at the time they are used in the process of the invention. However, the presence of water lowers the melting temperature of the new compound and that of the excess vanillin.

In order to guarantee the robustness of the process and so as not to risk being dependent on slight variations in the initial moisture content of the vanillin and ethyl vanillin powders, a variant of the process consists in intentionally adding from 1 to 5% of water to the mixture during the melting step.

The melting temperature will then be selected between 50° C. and 55° C. in order to keep the excess vanillin in solid form dispersed in the molten mixture, and the annealing operation previously described will become essential for drying the final product.

The process of the invention makes it possible to easily obtain a composition comprising essentially the new compound of vanillin and ethyl vanillin which has improved storage properties since the caking phenomenon is greatly reduced as indicated in the examples.

The melting point determined by differential scanning calorimetry varies slightly depending on the initial moisture content of the powder.

It is between 58 and 60° C. for dry powders (less than 0.1% by weight of water) and between 59 and 62° C. for powders with a higher moisture content (less than 2% by weight of water).

The X-ray diffraction spectrum of the composition obtained has the characteristic lines at angles 2θ (°)=20.7-25.6-27.5-28.0, as shown in FIG. 1, and which distinguish it from the spectra of vanillin and of ethyl vanillin.

With regard to its flowability properties, the composition of the invention has a flowability index after 24 hours of storage at 40° C. under air at 80% relative humidity, at a normal stress of 2 400 Pa, ranging between 0.05 and 0.6.

The process of the invention applies to vanillin and ethyl vanillin produced by any chemical synthesis, regardless of the starting substrate.

It is also suitable for vanillin obtained according to biochemical processes, in particular processes of microbiological fermentation, especially ferulic acid.

The invention does not exclude the use of one or more excipients with the composition of the invention.

It should be noted that the choice of the excipient(s) must take into account the intended use of the final product and therefore it must be edible if it is used in the food sector.

The amount of excipient(s) can be very variable and it can represent from 0.1 to 90% of the weight of the final mixture.

It is advantageously selected between 20 and 60% by weight.

Depending on the type of excipient selected, the amount used and the intended use of the final product, the excipient can be either added by dry mixing with the composition of the invention, or incorporated into the method for obtaining the composition of the invention, for example during the step of melting the mixture of vanillin and ethyl vanillin.

Examples of excipients that can be used are given hereinafter, but are given without being limiting in nature.

Fatty substances represent a first type of excipient.

As examples, mention may be made of fatty acids optionally in the form of salts or esters.

The fatty acids used are generally long-chain saturated fatty acids, i.e. fatty acids having a chain length between approximately 9 and 21 carbon atoms, such as, for example, capric acid, lauric acid, tridecylic acid, myristic acid, palmitic acid, stearic acid or behenic acid.

It is possible for said acids to be in salified form and mention may in particular be made of calcium stearate or magnesium stearate.

As fatty acid esters, mention may in particular be made of glyceryl stearate, isopropyl palmitate, cetyl palmitate and isopropyl myristate.

Mention may also more specifically be made of esters of glycerol and of long-chain fatty acids, such as glyceryl monostearate, glyceryl monopalmitostearate, glyceryl palmitostearate, ethylene glycol palmitostearate, polyglyceryl palmitostearate, polyglycol 1500 and 6000 palmitostearate, glyceryl monolinoleate; optionally mono- or diacetylated glycerol esters of long-chain fatty acids, such as monoacetylated or diacetylated monoglycerides and mixtures thereof; semisynthetic glycerides.

It is also possible to add a fatty alcohol of which the chain of carbon atoms is between approximately 16 and carbon atoms, such as, for example, myristyl alcohol, palmityl alcohol or stearyl alcohol.

It is also possible to use polyoxyethylenated fatty alcohols resulting from the condensation of linear or branched fatty alcohols, having from 10 to 20 carbon atoms, with ethylene oxide in a proportion from 6 to 20 mol of ethylene oxide per mole, such as, for example, coconut alcohol, tridecanol or myristyl alcohol.

Mention may also be made of waxes such as microcrystalline waxes, white wax, carnauba wax or paraffin.

Mention may be made of sugars, for instance glucose, sucrose, fructose, galactose, ribose, maltose, sorbitol, mannitol, xylitol, lactitol, maltitol; invert sugars: glucose syrups and also sucroglycerides derived from fatty oils such as coconut oil, palm oil, hydrogenated palm oil and hydrogenated soybean oil; sucrose esters of fatty acids, such as sucrose monopalmitate, sucrose monodistearate and sucrose distearate.

As examples of other excipients, mention may be made of polysaccharides, and mention may be made, inter alia, of the following products and mixtures thereof:
native, pregelatinized or modified starches derived in particular from wheat, corn, barley, rice, cassava or potato, and more particularly native corn starches rich in amylose, pregelatinized corn starches, modified corn starches, modified waxy corn starches, pregelatinized waxy corn starches, modified waxy corn starches, in particular the OSSA/sodium octenylsuccinate starch,
starch hydrolysates,
dextrins and maltodextrins resulting from the hydrolysis of a starch (wheat, corn) or of a potato flour, and also β-cyclodextrins,
cellulose, ethers thereof, in particular methyl cellulose, ethyl cellulose, methylethyl cellulose, hydroxypropyl cellulose; or esters thereof, in particular carboxymethyl cellulose or carboxyethyl cellulose optionally in sodium-containing form,
gums, such as carrageenan gum, Kappa-carrageenan or

Iota-carrageenan gum, pectin, guar gum, locust bean gum, xanthan gum, alginates, gum arabic, acacia gum, agar-agar.

A maltrodextrin having a degree of hydrolysis measured by “dextrose equivalent”, or DE, of less than 20 and preferably between 5 and 19, and more preferentially between 6 and 15, is preferentially selected.

As other excipients, mention may be made of flours, in particular wheat flour (native or pregel); starches, more particularly potato flour, arrowroot starch, corn starch, cornflour, sago or tapioca.

By way of excipients, use may also be made of gelatin (preferably having a gelling strength using a gelometer of 100, 175 and 250 Bloom). It can without distinction come either from acid treatment of pig skin and ossein, or from alkaline treatment of cowhide and ossein.

It is also possible to add other excipients, such as silica or else, for example, an antioxidant such as, in particular, vitamin E or an emulsifier, in particular lecithin.

In order to adjust the flavoring power of the mixture or enhance its taste, the use of ethylmaltol and/or of propenyl guaethol can be envisioned.

The invention does not exclude the addition of a supplementary amount of vanillin or ethyl vanillin.

The choice of the excipients is made as previously mentioned according to the application envisioned.

The composition of the invention can also be used in many fields of application, inter alia, in the food and pharmaceutical sector, and in the perfumery industry.

A preferred field of application of the use of the composition of the invention is in the cookie trade and cake-making industry, and more particularly:
dry cookie trade: sweet cookies of conventional type, butter cookies, large round cookies, snacks, shortbread,
factory-baked cakes: champagne ladyfingers, thin fingers, sponge fingers, Genoa cake, sponge cake, madeleines, pound cakes, fruit cakes, almond cakes, petit fours.

The essential elements present in the mixtures intended for the abovementioned industries are proteins (gluten) and starch, which are most commonly provided by wheat flour. For preparing the various types of cookies and cakes, ingredients such as sucrose, salt, eggs, milk, fat, optionally chemical yeasts (sodium bicarbonate or other artificial yeasts) or biological yeasts and flours from various cereals, etc. are added to the flour.

The composition according to the invention is incorporated during the manufacture, depending on the desired product, according to conventional techniques in the field under consideration (cf. in particular J. L. Kiger and J. C. Kiger—Techniques Modernes de la Biscuiterie, Pâtisserie-Boulangerie industrielles et artisanales [Modern techniques of industrial and traditional production of cookies, cakes and bakery products], DUNOD, Paris, 1968, Volume 2, pp. 231 ff.).

Preferentially, the composition of the invention is introduced into the fats which are used in the preparation of the dough.

By way of indication, it will be specified that the composition of the invention is introduced in an amount of from 0.005 to 0.2 g per kg of dough.

The composition of the invention is perfectly suitable for use in the chocolate-making field, regardless of the form in which it is used: bars of chocolate, couverture chocolates, filling for chocolates.

It can be introduced during conching, i.e. blending of the cocoa paste with the various ingredients, in particular flavorings, or after conching, by processing in the cocoa butter.

In this field of application, the composition of the invention is used, depending on the type of chocolate, in a proportion of from 0.0005 g to 0.1 g per 1 kg of final product: the highest contents being used in couverture chocolate.

Another use of the composition of the invention is the manufacture of candies of all kinds: sugared almonds, caramels, nougats, hard candies, fondant candies and the like.

The amount of the composition of the invention introduced depends on the more or less strong taste that is desired. Thus, the doses of use of the compound of the invention can range between 0.001% and 0.2%.

The composition of the invention is very suitable for uses in the dairy industry, and more particularly in flavored and gelled milks, cream desserts, yoghurts, ices and ice creams.

The flavoring is carried out by simple addition of the composition of the invention, in one of the mixing stages required during production of the product.

The contents of said composition to be used are generally low, about 0.02 g per 1 kg of final product.

Another application of the composition of the invention in the food sector is the preparation of vanillin sugar, i.e. impregnation of sugar with vanillin, in a content of about 7 g expressed relative to 1 kg of final product.

The composition of the invention can also be included in various drinks, and mention may be made, inter alia, of grenadine and chocolate drinks.

In particular, it can be used in preparations for instant drinks delivered by automatic drinks dispensers, flavored drinks powders, chocolate powder or else in instant preparations in the form of powder intended for making desserts of all kinds, custard tarts, cake mixtures, pancakes, after dilution with water or with milk.

It is common practice to use vanillin for denaturing butter. To this effect, the composition of the invention can be used in a proportion of 6 g per metric tonne of butter.

Another field of application of the composition of the invention is animal feed, in particular for preparing meal for calf and pig feeds. The recommended content is approximately 0.2 g per kg of meal to be flavored.

The composition of the invention can find other applications, such as a masking agent, for the pharmaceutical industry (for masking the odor of a medicament) or for other industrial products (such as gum, plastic, rubber, etc.).

It is entirely suitable in completely different fields such as the cosmetics industry, the perfumery industry or the detergent industry.

It can be used in cosmetics such as creams, milks, make-up and other products, and also, as fragrancing ingredients, in fragrancing compositions and fragranced substances and products.

The term “fragrancing compositions” denotes mixtures of various ingredients such as solvents, solid or liquid carriers, fixing agents, various odorous compounds, etc., into which the composition of the invention is incorporated and is used to give the desired fragrance to various types of final product.

Fragrance bases constitute preferred examples of the fragrancing compositions in which the composition of the invention can advantageously be used at a content of from 0.1% to 2.5% by weight.

The fragrance bases can be used for preparing numerous fragranced products, such as, for example, eaux de toilettes [toilet waters], fragrances, aftershave lotions; toiletries and hygiene products, such as bath or shower gels, deodorant or antiperspirant products, whether in the form of sticks or lotions, talcs or powders of any nature; products for the hair, such as shampoos and hair products of any type.

Another example of use of the composition of the invention is the soap-making field. It can be used in a content of from 0.3% to 0.75% of the total mass to be fragranced. Generally, it is combined, in this application, with benzoin resinoid and sodium hyposulfite (2%).

The composition according to the invention can find many other applications, in particular in air fresheners or any maintenance product.

The physicochemical characteristics of the compositions of the invention are determined according to the following methods:

### 1. Melting Point

The melting point of the composition of the invention is measured by differential scanning calorimetry.

The measurement is carried out using a Mettler DSC822e differential scanning calorimeter under the following conditions:
preparation of the sample at ambient temperature: weighing out and introduction into a sample carrier,
sample carrier: crimped aluminum capsule,
test specimen: 8.4 mg,
rate of temperature increase: 2° C./min,
study range: 10-90° C.

The sample of the composition is weighed out and introduced into the capsule, which is crimped and then placed in the apparatus.

The temperature program is run and the melting profile is obtained on a thermogram.

The melting temperature is defined on the basis of a thermogram produced under the above operating conditions.

The onset temperature is retained: temperature corresponding to the maximum slope of the melting peak.

### 2. X-Ray Diffraction Spectrum

The X-ray diffraction spectrum of the composition of the invention is determined using the X′Pert Pro MPD PANalytical apparatus equipped with an X′ Celerator detector, under the following conditions:
Start Position [°2Th.]: 1.5124
End Position [°2Th.]: 49.9794
Step Size [°2Th.]: 0.0170
Scan Step Time [s]: 41.0051
Anode Material: Cu
K-Alpha1 [Å]: 1.54060
Generator Settings: 30 mA, 40 kV

### 3. Flowability Property and Caking Index

The composition of the invention has the characteristic of caking less on storage, which is demonstrated by determining the degree of flowability of the powder.

The flowability of powders is a technical notion well known to those skilled in the art. For further details, reference may be made in particular to the handbook “Standard shear testing technique for particulate solids using the Jenike shear cell”, published by “The Institution of Chemical Engineers”, 1989 (ISBN: 0 85295 232 5).

The flowability index is measured in the following way.

The flowability of powders is measured by shearing a sample in an annular cell (sold by D. Schulze, Germany).

The preshearing of the powders is carried out under a normal stress of 5200 Pa.

The shear points necessary for plotting the yield locus of the sample are obtained for four normal stresses below the stress of the preshearing, typically 480 Pa, 850 Pa, 2050 Pa and 3020 Pa.

From the Mohr circles in the diagram of “shear stress as a function of normal stresses”, two stresses are determined on the yield locus, which stresses characterize the sample:
the normal stress in the main direction; it is given by the end of the large Mohr circle which passes through the preshear point,
the cohesive force; it is given by the end of the small Mohr circle which is tangent to the yield locus and passes through the origin.

The ratio of the normal stress in the main direction to the cohesive force is a dimensionless number, referred to as “i, flowability index”.

These measurements are carried out immediately after filling the annular cell; the immediate flowability index is thus obtained.

Another series of measurements is carried out with a cell which has been stored for 24 hours at 40° C. and 80% relative humidity under a normal stress of 2400 Pa.

The caking index is thus obtained.

Examples illustrating the present invention, without being limiting in nature, are given hereinafter.

In the examples, the percentages mentioned are expressed by weight.

### EXAMPLE 1

350 g of powdered vanillin (VA) and 150 g of powdered ethyl vanillin (EVA), i.e. a VA/EVA weight ratio of 70/30, are introduced into a stirred reactor equipped with heating via a double jacket. The moisture content of these powders is 0.1% by weight.

This mixture is brought to 63° C. with stirring.

A suspension of very fine particles of vanillin dispersed in a molten mixture of vanillin+ethyl vanillin is thus obtained.

This suspension is poured onto a stainless steel plate kept at 50° C., so as to form thereon a thin film approximately 1 mm thick.

The crystallization is complete in less than one minute.

The resulting solid sheet is easily detached from the stainless steel; it is left at ambient temperature until cooling is complete.

This sheet is then coarsely crushed so as to be able to feed an oscillating-arm granulator (Erweka FGS granulator) fitted with a screen with a mesh size of 1.0 mm.

The product is moderately milled therein so as to give granules, the size of which ranges from 0.1 to 1.0 mm.

The melting point of the granules is determined by differential scanning calorimetry as previously described. The thermogram obtained shows a main peak which corresponds to the new vanillin/ethyl vanillin compound. The melting temperature (Tonset) which corresponds to the maximum slope of the peak is 60° C.

The X-ray diffraction spectrum of the granules exhibits the characteristic lines at angles 2θ (°)=20.7-25.6-27.5-28.0, as shown in FIG. 1, and which distinguish it from the vanillin and ethyl vanillin spectra.

The granules, stored for one month at 22° C. in a one liter glass bottle, still exhibit good flowability.

By way of comparison, a mixture of the two powders of vanillin and ethyl vanillin, stored under the same conditions, is completely set after one week, regardless of the VA/EVA weight ratio of between 2/98 and 98/2.

### EXAMPLE 2

278 g of powdered vanillin and 150 g of powdered ethyl vanillin, i.e. a VA/EVA weight ratio=65/35, are introduced into a stirred reactor equipped with heating via a double jacket. The moisture content of these powders is 0.1% by weight.

This mixture is brought to 62° C. with stirring.

After about ten minutes, melting is complete and a homogeneous translucent liquid is obtained.

72 g of powdered vanillin are added and dispersed in the liquid by means of stirring.

The resulting suspension is poured onto a stainless steel plate kept at 20° C., so as to form thereon a thin film approximately 1 mm thick.

The crystallization is complete in a few seconds.

The resulting solid sheet is easily detached from the stainless steel; it is coarsely crushed so as to be able to feed an oscillating-arm granulator (Erweka FGS granulator) fitted with a screen with a mesh size of 1.0 mm.

The product is moderately milled therein so as to give granules, the size of which ranges from 0.1 to 1.0 mm.

The granules are introduced into a powder mixer equipped with heating via a double jacket. The temperature, initially 20° C., is gradually increased to reach 52° C. in the mass of the granules. The time taken to bring the granules to temperature is approximately 30 minutes. The granules are kept at 52° C. with stirring for two hours.

The resulting granules have a melting point of 61° C. measured by differential scanning calorimetry (Tonset).

The X-ray diffraction spectrum of the granules exhibits the characteristic lines at angles 2θ (°)=20.7-25.6-27.5-28.0, as shown in FIG. 1, and which distinguish it from the vanillin and ethyl vanillin spectra.

The granules, stored for one month at 22° C. in a one liter glass bottle, still exhibit good flowability.

By way of comparison, a mixture of the two powders of vanillin and ethyl vanillin, stored under the same conditions, is completely set after one week, regardless of the VA/EVA weight ratio of between 2/98 and 98/2.

### EXAMPLE 3

350 g of powdered vanillin and 150 g of powdered ethyl vanillin, i.e. a VA/EVA weight ratio=70/30, are introduced into a stirred reactor equipped with heating via a double jacket and then 17 g of water, i.e. 3.4% of the total weight of solid, are added.

This mixture is brought to 55° C. with stirring.

A suspension of very fine particles of the vanillin dispersed in a molten mixture of vanillin+ethyl vanillin+water, is thus obtained.

The suspension is poured onto a stainless steel plate kept at 50° C., so as to form thereon a thin film approximately 1 mm thick. The crystallization is complete after approximately 5 minutes.

The resulting solid sheet is easily detached from the stainless steel; it is left at ambient temperature until cooling is complete.

This sheet is then coarsely crushed so as to be able to feed an oscillating-arm granulator (Erweka FGS granulator) fitted with a screen with a mesh size of 1.0 mm.

The product is moderately milled therein so as to give granules, the size of which ranges from 0.1 to 1.0 mm.

The granules are introduced into a powder mixer equipped with heating via a double jacket.

The temperature, initially 20° C., is gradually increased to reach 52° C. in the mass of the granules.

The time taken to bring the granules to temperature is approximately 30 minutes.

The granules are kept at 52° C. with stirring for two hours.

Throughout this operation, the roof of the mixer is swept with a dry nitrogen stream in order to expel the water vapor released by the granules.

The resulting granules have a melting point of 61° C. measured by differential scanning calorimetry (Tonset).

The X-ray diffraction spectrum of the granules exhibits the characteristic lines at angles 2θ (°)=20.7-25.6-27.5-28.0, as shown in FIG. 1, and which distinguish it from the vanillin and ethyl vanillin spectra.

The immediate flowability index and the flowability index after 24 hours of storage at 40° C. under air at 80% relative humidity under a normal stress of 2400 Pa were determined using an annular shear cell in accordance with the method previously described.

The results recorded in table (I) given hereinafter in example 4 make it possible to note that the granules obtained according to the process of the invention have a flowability after storage under stress which is comparable to the powders of pure vanillin or of pure ethyl vanillin and much greater than a mixture of these two powders.

### EXAMPLE 4

278 g of powdered vanillin and 150 g of powdered ethyl vanillin, i.e. a VA/EVA weight ratio=65/35, are introduced into a stirred reactor equipped with heating via a double jacket, and then 17 g of water, i.e. 4.0% of the weight of solid, are added.

This mixture is brought to 51° C. with stirring.

After about ten minutes, melting is complete and a homogeneous translucent liquid is obtained.

72 g of powdered vanillin are then added and dispersed in the liquid by means of stirring.

The resulting suspension is poured onto a stainless steel plate kept at 20° C. so as to form thereon a thin film approximately 1 mm thick.

The crystallization is complete in a few seconds.

The resulting solid sheet is easily detached from the stainless steel; it is coarsely crushed so as to be able to feed an oscillating-arm granulator (Erweka FGS granulator) fitted with a screen with a mesh size of 1.0 mm.

The product is moderately milled therein to give granules, the size of which ranges from 0.1 to 1.0 mm.

The granules are introduced into a powder mixer equipped with heating via a double jacket.

The temperature, initially 20° C., is gradually increased to reach 52° C. in the mass of the granules.

The time taken to bring the granules to temperature is approximately 30 minutes.

The granules are kept at 52° C. with stirring for two hours.

Throughout this operation, the roof of the mixer is swept with a dry nitrogen stream in order to expel the water vapor released by the granules.

The resulting granules have a melting point of 59° C. measured by differential scanning calorimetry (Tonset).

The X-ray diffraction spectrum of the granules exhibits the characteristic lines at angles 2θ (°)=20.7-25.6-27.5-28.0, as shown in FIG. 1, and which distinguish it from the vanillin and ethyl vanillin spectra.

The immediate flowability index and the flowability index after 24 hours of storage at 40° C. under air at 80% relative humidity under a normal stress of 2400 Pa were determined using an annular shear cell in accordance with the method previously described.

The results recorded in table (I) make it possible to note that the granules obtained according to the process of the invention have a flowability after storage under stress which is much greater than a dry mixture of these two powders.

TABLE I

| Nature of the | Immediate | Flowability index |
|---|---|---|
| product | flowability index | after storage* |
| Vanillin powder | 5.6 | 0.66 |
| (comparative) | | |
| Ethyl vanillin | 6.5 | 0.61 |
| powder | | |
| (comparative) | | |
| Mixture of | 40 | 0.03 |
| vanillin and | | |
| ethyl vanillin | | |
| powders 70/30 by | | |
| weight | | |
| (comparative) | | |
| Granules of | 3.6 | 0.35 |
| example 3 | | |
| (invention) | | |
| Granules of | 22 | 0.10 |
| example 4 | | |
| (invention) | | |

*= storage at 40° C. under air at 80% relative humidity under a normal stress of 2400 Pa.

### EXAMPLE 5

In this example, a composition is prepared in the form of granules comprising 50% by weight of the granules prepared according to example 3 and 50% by weight of sucrose.

The mixing operation, which lasts approximately 5 min, is carried out at ambient temperature in a WAM plow mixer.

### EXAMPLE 6

In this example, a composition is prepared in the form of granules comprising 50% by weight of the granules prepared according to example 3 and 5% by weight of a maltodextrin (Roquette Glucidex IT6).

The mixing operation, which lasts approximately 5 min, is carried out at ambient temperature in a WAM plow mixer.

The immediate flowability index and the flowability index after 24 hours of storage at 40° C. under air at 80% relative humidity under a normal stress of 2400 Pa were determined using an annular shear cell in accordance with the method previously described.

The results are recorded in table (II).

TABLE II

| Nature of the | Immediate | Flowability index |
|---|---|---|
| product | flowability index | after storage* |
| Vanillin powder | 5.6 | 0.66 |
| (comparative) | | |
| Ethyl vanillin | 6.5 | 0.61 |
| powder | | |
| (comparative) | | |
| Mixture of | 40 | 0.03 |
| vanillin and | | |
| ethyl vanillin | | |
| powders 70/30 by | | |
| weight | | |
| (comparative) | | |
| Composition of | 15 | 0.11 |
| example 5 | | |
| (invention) | | |
| Composition of | 30 | 0.50 |
| example 6 | | |
| (invention) | | |

*= storage at 40° C. under air at 80% relative humidity under a normal stress of 2400 Pa.

It is noted that the compositions obtained according to the process of the invention have a caking index after storage under stress which is highly superior to that of a simple dry mixture of the vanillin and ethyl vanillin powders.

In a 50/50 mixture by weight with a maltodextrin, these compositions have a caking index which is comparable to that of the powders of pure vanillin or pure ethyl vanillin.

1. A process for preparing a composition, the process comprising:
melting an initial mixture of vanillin and ethyl vanillin, in a molar ratio other than 2, with an excess of vanillin representing from 2% to 20% by weight of the mixture to provide a molten mixture, wherein a melting temperature is selected such that a new compound obtained is completely molten and excess vanillin remains in a solid state finely dispersed in a molten mixture in order to act as crystallization seeds,
solidifying said mixture by cooling to a temperature of less than or equal to 50° C.±1° C.,
recovering a resulting composition comprising the new compound, and
optionally, heat treating to a temperature of 51° C.±1° C., wherein the resulting composition is comprised of a compound based on vanillin and ethyl vanillin in a vanillin/ethyl vanillin molar ratio of 2.

2. The process as defined by claim 1, wherein the initial mixture of vanillin and the ethyl vanillin are in the following proportions:
from 67% to 72% by weight of vanillin, and
from 28% to 33% by weight of ethyl vanillin.

3. The process as defined by claim 1, wherein the initial mixture of vanillin and ethyl vanillin are used in the following proportions:
from 67% to 70% by weight of vanillin, and
from 30% to 33% by weight of ethyl vanillin.

4. The process as defined by claim 1, wherein the vanillin and the ethyl vanillin are loaded separately or as a mixture and the mixture is brought to a temperature ranging from 62° C. to 70° C.

5. The process as defined by claim 1, wherein preparation of the molten mixture is carried out under an atmosphere of inert gas.

6. The process as defined by claim 1, wherein cooling of the molten mixture is carried out in the absence of stirring, resulting in a solidified composition comprising the new compound of vanillin and ethyl vanillin.

7. The process as defined by claim 1, the process further comprising crystallizing the molten mixture at any temperature below 50° C. recovering the solid obtained, and then subjecting it to an annealing operation.

8. The process as defined by claim 7, wherein the annealing is carried out by gradually bringing the solid obtained to a temperature of 51° C.±1 and keeping it at this temperature for several minutes.

9. The process as defined by claim 7, wherein the annealing is carried out with stirring.

10. The process as defined by claim 1, the process further comprising adding from 1% to 5% of water to the mixture during the melting step, wherein the melting temperature is selected to range from 50° C. to 55° C., and carrying out the annealing operation.

11. The process as defined by claim 1, wherein the composition obtained is formed according to a milling technique.

12. The process as defined by claim 1, wherein the composition obtained is formed according to a flake-forming, prilling or spray-cooling technique.

13. A composition comprising:
from 80% to 99% by weight of a mixture of the new vanillin/ethyl vanillin compound and of vanillin, and
from 1% to 20% by weight of other crystalline phases.

14. The composition as defined by claim 13, comprising:
from 90% to 99% by weight of a mixture of the new vanillin/ethyl vanillin compound and of vanillin, and
from 1% to 10% by weight of other crystalline phases.

15. The composition as defined by claim 13, wherein the mixture obtained comprises:
from 80% to 94% by weight of the new vanillin/ethyl vanillin compound, and
from 6% to 20% by weight of vanillin.

16. The composition as defined by claim 15, wherein the mixture obtained comprises:
from 86% to 94% by weight of the new vanillin/ethyl vanillin compound, and
from 6% to 14% by weight of vanillin.

17. A composition comprising at least one composition as defined by claim 13, and at least one excipient wherein said at least one excipient is selected from a fatty substance; a fatty alcohol; a sugar; a polysaccharide; a silica; a vanillin and an ethyl vanillin.

18. The composition as defined by claim 17, wherein the excipient is selected from the group consisting of:
sugar; an invert sugar: a sucrose ester of a fatty acid,
a native, pregelatinized or modified starch,
a starch hydrolysate,
a dextrin or maltodextrin resulting from hydrolysis of a starch, and also a β-cyclodextrin,
a cellulose, an ester thereof, or an ester thereof,
a gum,
a flour,
a gelatin,
a silica,
an antioxidant,
an emulsifier, and
a vanillin or an ethyl vanillin.

19. The composition as defined by claim 17, wherein the composition comprises from 0.1% to 90% by weight of excipient(s).

20. A method of using the composition as defined by claim 13, the method comprising using the composition as a flavoring in human food or animal feed, in a pharmaceutical, in a fragrance, in a cosmetic, in a perfume or in a detergent.

21. The method as defined by claim 20, comprising using the composition during manufacture of a dough, in chocolate-making, during manufacture of candies, in a dairy product, in preparation of vanillin sugar, in preparation of a drink, in preparation of an instant drink, in an instant preparation in the form of powder or for denaturing butter.

22. The method as defined by claim 20, comprising using the composition in animal feed.

23. The method as defined by claim 20, comprising using the composition as an odor-masking agent.

24. The process as defined by claim 4, wherein the temperature ranges from 62° C. to 65° C.

25. The process as defined by claim 5, wherein the inert gas is nitrogen.

26. The process as defined by claim 7, wherein the temperature for crystallizing the molten mixture ranges from 20° C. to 50° C. (limit excluded).

27. The composition as defined by claim 18, wherein the sugar is selected from the group consisting of glucose, sucrose, fructose, galactose, ribose, maltose, sorbitol, mannitol, xylitol, lactitol and maltitol.

28. The composition as defined by claim 18, wherein the inert sugar is selected from the group consisting of a glucose syrup or a sucroglyceride derived from a fatty oil.

29. The composition as defined by claim 28, wherein the fatty oil is selected from the group consisting of coconut oil, palm oil, hydrogenated palm oil and hydrogenated soybean oil.

30. The composition as defined by claim 18, wherein the sucrose esters of the fatty acid is a sucrose monopalmitate, a sucrose monodistearate or a sucrose distearate.

31. The composition as defined by claim 18, wherein the starch is derived from wheat, corn, barley, rice, cassava or potato.

32. The composition as defined by claim 31, wherein the starch is derived from a native corn starches rich in amylose, a pregelatinized corn starch, a modified corn starch, a modified waxy corn starch, a pregelatinized waxy corn starch, or a modified waxy corn starch.

33. The composition as defined by claim 32, wherein the starch is a OSSA/sodium octenylsuccinate starch.

34. The composition as defined by claim 18, wherein the starch is from wheat, corn or potato flour.

35. The composition as defined by claim 18, wherein the maltodextrin has a DE of less than 20.

36. The composition as defined by claim 35, wherein the maltodextrin has a DE of from 5 to 19.

37. The composition as defined by claim 36, wherein the maltodextrin has a DE of from 6 to 15.

38. The composition as defined by claim 18, wherein the cellulose is a methylethyl cellulose, an ethyl cellulose, a methyl ethyl cellulose or an hydroxypropyl cellulose.

39. The composition as defined by claim 18, wherein the cellulose ester is a carboxymethyl cellulose, or a carboxyethyl cellulose optionally in a form comprising sodium.

40. The composition as defined by claim 18, wherein the gum is a carrageenan gum, a Kappa-carrageenan, a Iota-carrageenan gum, a pectin, a guar gum, a locust bean gum, a xanthan gum, an alginate, a gum arabic, an acacia gum or an agar-agar.

41. The composition as defined by claim 18, wherein, the flour is a wheat flour (native or pregel) or a starch.

42. The composition as defined by claim 41, wherein the flour is a potato flour.

43. The composition as defined by claim 18, wherein the antioxidant is vitamin E.

44. The composition as defined by claim 18, wherein the emulsifier is lecithin.

45. The composition as defined by claim 19, wherein the composition comprises from 20% to 60% by weight of excipients.
